(19) United States
(12) Patent Application Publication
(10) Pub. No.: US 2025/0204903 A1
(43) Pub. Date: Jun. 26, 2025

(54) BELT ADJUSTMENT DEVICES

(71) Applicant: Nicole Monique Atondo, Minneapolis, MN (US)

(72) Inventor: Nicole Monique Atondo, Minneapolis, MN (US)

(21) Appl. No.: 18/989,976

(22) Filed: Dec. 20, 2024

Related U.S. Application Data
(60) Provisional application No. 63/614,282, filed on Dec. 22, 2023.

(51) Int. Cl. A61B 17/02; A61B 17/00

(52) U.S. Cl. CPC A61B 17/02; A61B 2017/00407; A61B 2017/00951; A61B 2017/0287

(57) ABSTRACT

A device may include a belt. A device may include an adjustment mechanism to adjust the distance of the belt between an end of the belt and the adjustment mechanism. A device may include an adhesive material coupled to the end of the belt to adhere to a portion of skin of a human user, wherein the adjustment mechanism adjusts a distance between the portion of skin of the human user and the adjustment mechanism.

### PRIORITY INFORMATION

This application claims benefit of Provisional Application Ser. No. 63/614,282, filed Dec. 22, 2023, the contents of which are incorporated herein by reference.

### TECHNICAL FIELD

Embodiments of the disclosure relate generally to belt devices that can be utilized to separate folds of human skin to prevent contact between two portions of human skin.

### BACKGROUND

Patients in the area of medicine can develop bedsores. Bedsores, also known as pressure ulcers or pressure sores, are localized injuries to the skin and underlying tissue, usually over bony prominences, caused by prolonged pressure and/or friction. They most commonly develop in individuals who are immobile or have limited mobility, such as those who are bedridden, wheelchair-bound, or have difficulty changing positions. The formation of bedsores typically follows a series of stages: pressure, reduced blood flow, friction and shear, tissue damage, and/or inflammation.

Some patients can be incapacitated in the area of medicine. These incapacitated patients may not be able to separate buttocks muscles for cleaning or other procedures. These types of patients can be difficult to monitor and/or perform procedures on for medical personnel. Current systems can be difficult to utilize and/or uncomfortable for a human user to utilize.

### BRIEF DESCRIPTION OF THE DRAWINGS

The present disclosure will be understood more fully from the detailed description given below and from the accompanying drawings of various embodiments of the disclosure.

FIG. 1 illustrates an example of a belt adjustment device according to examples described herein.

FIG. 2 illustrates an example of a belt adjustment device according to examples described herein.

FIG. 3 illustrates an example of a belt adjustment device according to examples described herein.

FIG. 4 illustrates an example of a system that includes belt adjustment device utilized with an additional medical device according to examples described herein.

FIG. 5 illustrates an example of a system that includes belt adjustment device utilized with an additional medical device according to examples described herein.

FIG. 6 illustrates an example of a belt adjustment device according to examples described herein.

FIG. 7 illustrates an example of a belt adjustment device according to examples described herein.

### DETAILED DESCRIPTION

An individual or user (e.g., human user, animal, etc.) can be in a state of health where areas of skin or skin surface is in contact with other areas of skin for extended periods of time. In these examples, the user and/or caretaker of the user may want to separate the two areas of skin for an extended period of time to allow the two areas of skin or area between the two areas of skin to dry or be cleaned for the extended period of time. For example, a user can experience bed sores or other epidermis issues caused by prolonged contact between areas of skin. As used herein, bed sores, also known as pressure ulcers or decubitus ulcers, are injuries to the skin and underlying tissues caused primarily by prolonged pressure. They typically occur in individuals who are immobile, bedridden, or wheelchair-bound for extended periods.

In these examples, a belt adjustment device as described herein can be utilized to separate the two areas of skin to allow access to the area of skin between the two areas of skin. For example, the belt adjustment device can be utilized to separate a first buttocks from a second buttocks of a user to allow access to the area between the two buttocks. In this way, the area between the two buttocks can be cleaned or dried after cleaning. In this way, the area between the first area of skin and the second area of skin can be exposed to the elements and avoid skin irritation such as, but not limited to, bedsores and rashes.

The present disclosure relates to belt adjustment devices. In some aspects, the techniques described herein relate to a device, including: a belt, an adjustment mechanism to adjust the distance of the belt between an end of the belt and the adjustment mechanism; an adhesive material coupled to the end of the belt to adhere to a portion of skin of a human user, where the adjustment mechanism adjusts a distance between the portion of skin of the human user and the adjustment mechanism. In some aspects, the techniques described herein relate to a device, wherein the adhesive material is removable from the end of the belt.

In some aspects, the techniques described herein relate to a device, wherein the adhesive material is coupled to the end of the of the belt by a connection interface. In some aspects, the techniques described herein relate to a device, wherein the connection interface is a system of hooks and loops. In some aspects, the techniques described herein relate to a device, wherein the adjustment mechanism is a ratcheting mechanism to adjust the distance of the belt incrementally.

In some aspects, the techniques described herein relate to a device, including: a belt, an adjustment mechanism to adjust a distance of the belt between a first end and a second end, a first adhesive material coupled to the first end of the belt to adhere to a first portion of skin of a human user, and a second adhesive material coupled to the second end of the belt to adhere to a second portion of skin of human user; wherein the adjustment mechanism adjusts a distance between the first portion of skin of the human user and the second portion of skin of the human user. In some aspects, the techniques described herein relate to a device, wherein the first adhesive material coupled to the belt is applied directly to the first end and the second adhesive material coupled to the belt is applied directly to the second end of the belt.

In some aspects, the techniques described herein relate to a device, wherein the first adhesive material is applied to a first disposable surface and the second adhesive material is applied to a second disposable surface. In some aspects, the techniques described herein relate to a device, wherein the first end and second end include a hooking apparatus to interact with the skin of the human user. In some aspects, the techniques described herein relate to a device, including: a belt; an adjustment mechanism to adjust a distance of the belt between a first end and a second end; a first set of protrusions coupled to the first end of the belt to interact with a first portion of skin of a human user; and a second set of protrusions coupled to the second end of the belt to interact with a second portion of skin of the human user; wherein the adjustment mechanism adjusts a distance between the first portion of skin of the human user and the second portion of skin of the human user.

In some aspects, the techniques described herein relate to a device, further including a first plurality of caps to cover the first set of protrusions and a second plurality of caps to cover the second set of protrusions. In some aspects, the techniques described herein relate to a device, wherein the caps are attachable and removable from the protrusions.

In some aspects, the techniques described herein relate to a device, wherein the caps consist of an outer adhesive surface. In some aspects, the techniques described herein relate to a device, wherein the protrusions are no more than 4.5 inches in length. In some aspects, the techniques described herein relate to a device, wherein the adjustment mechanism includes an attachment mechanism to couple the adjustment mechanism to a base.

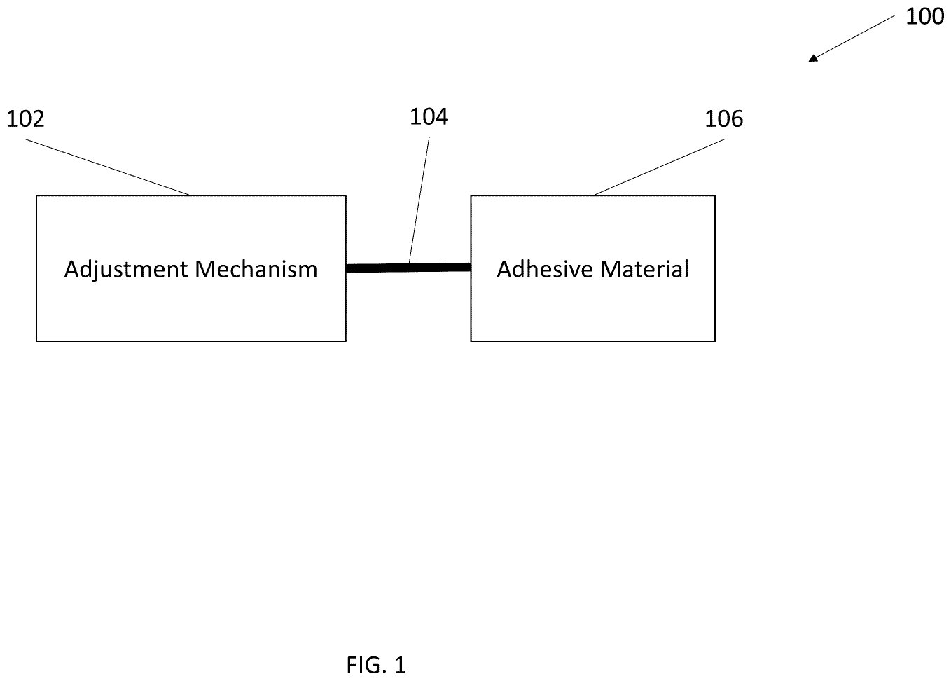

FIG. 1 illustrates an example of a belt adjustment device 100 according to examples described herein. The belt adjustment device 100 can include a belt material 104 between an adhesive material 106 and an adjustment mechanism 102. The adhesive material 106 can include an adhesive that is able to stick to human skin without damaging the skin. In some embodiments, the adhesive material 106 can be a medical adhesive that is safe for skin, but also able to maintain a location on the skin when the adjustment mechanism 102 applies pressure on the belt material 104.

In some embodiments, the belt material 104 can refer to medical belts or straps that are used for support, compression, stabilization, or restraint in various healthcare applications. They can be made from a range of materials depending on their purpose, comfort, durability, and flexibility requirements. The belt material 104 can be an elastic material such as Spandex, Lycra, Neoprene, or elastic blends. The clastic material can be a material that is stretchable and flexible to allow the belt material 104 to be flexed around a surface of the human user while still providing pressure between the adjustable mechanism 102 and the adhesive material 106.

In some embodiments, the belt material 104 can be a neoprene material such as, but not limited to a rubber or synthetic rubber. In other embodiments, the belt material 104 can be a cotton material that can be a woven belt to be less elastic than the elastic material or neoprene material. In some embodiments, the belt material 104 can be a nylon material, polyester material, leather, hook and loop (Velcro) components, silicone or gel-filled materials, polyurethane foam material, among other materials that can allow the adjustment mechanism 102 to provide pressure on the adhesive material 106 and move the skin portion that is coupled to the adhesive material 106. For example, the adjustment mechanism 102 can be connected to a medical chair or other anchor device and utilized to provide retention on the belt material 104 to move the adhesive material 106 toward the anchor device.

Various types of adhesives can be used on human skin for medical, cosmetic, or adhesive purposes. The choice of adhesive depends on the intended use, the duration of adhesion required, and any specific medical or skin sensitivities. Some common types of adhesives used on human skin include, but are not limited to: medical tape adhesives, surgical adhesives, ostomy adhesives, and/or cosmetic adhesives. The adhesive material 106 can include the adhesive that is located on a pad or substrate that can be connected or connectable to the belt material 104. The belt material can be an elastic material and/or fiber material that can allow the adjustment mechanism 102 to pull the adhesive material 106 and area of skin attached to the adhesive material 106 away from a different area of skin. The belt material 104 can be utilized to maintain a connection between the adhesive material and the adjustment mechanism 102 such that the adjustment mechanism 102 can provide pressure on the belt material 104 such that the pressure is transferred to the adhesive material 106.

In some embodiments, the adhesive material 106 can be a medical adhesive material. As used herein, medical adhesives can be designed for human skin and are formulated to be safe, hypoallergenic, and effective for securing dressings, devices, or other materials to the skin. These adhesives balance adhesion strength with skin safety, minimizing the risk of irritation or damage. The medical adhesive can include, but is not limited to: acrylic adhesives, silicone adhesives, hydrocolloid adhesives, zinc oxide adhesives, rubber based adhesives, hydrogel adhesives, and/or pressure-sensitive adhesives. In some specific examples, the medical adhesive can be strong enough to pull a portion of skin away from a different portion of skin. Some specific adhesive materials 106 can include Tegaderm™, Durapore™, Dermabond™, and/or other types of adhesives that have an adhesive shear strength or shear adhesion that is above a particular threshold.

As used herein, adhesive shear strength can refer to an ability of an adhesive to resist forces applied parallel to its surface (e.g., pulling or dragging forces). This measures how well the adhesive holds under stress without slipping or failing. Other factors can include, tensile adhesion, peel adhesion, cohesive strength, holding force, among other factors. In this way, the mass of the skin portion coupled to the adhesive material can be utilized to determine the threshold adhesive shear strength or threshold shear adhesion for the adhesive material 106 to provide a sufficient quantity of adhesion to alter the location of the skin portion coupled to the adhesive material 106.

As described further herein, the adjustment mechanism 102 can be utilized to alter a length of the belt material 104 and maintain the altered length. For example, the adjustment mechanism 102 can be a ratcheting device. For example, the adjustment mechanism 102 can be a mechanical tool or mechanism designed to allow motion in one direction while preventing motion in the opposite direction. The adjustment mechanism 102 can achieve this by using a system of teeth or pawls that engage with a gear or ratchet wheel. Although a ratcheting mechanism is described, other types of adjustment mechanisms can be utilized to alter the length of the belt material 104 and maintain a pressure on the adhesive material 106.

In some embodiments, the adjustment mechanism 102 be a standard buckle mechanism that utilizes a pin and hole system. In other embodiments, the adjustment mechanism 102 can be a D-ring buckle that utilizes two D-shaped rings to create a friction lock. In other embodiments, the adjustment mechanism 102 can be a quick-release buckle that utilizes a male-female interlocking clip where adjustments are made by pulling one end of the strap through a tensioner on the buckle.

In other embodiments, the adjustment mechanism 102 can be ratchet buckle where a toothed strap is fed through a rachet buckle. In this embodiment, the belt material 104 can include a toothed strap portion that is fed through the adjustment mechanism 102 (e.g., ratchet buckle, etc.). In other embodiments, the adjustment mechanism 102 can include a cam buckle adjuster where a cam lever holds the belt material 104 in place by clamping down on it. To adjust the length of the belt material 104, the cam is lifted and the belt material 104 is slid through the adjustment mechanism 102.

In other embodiments, the adjustment mechanism 102 is a sliding adjuster mechanism (tri-glide or ladder lock, etc.) that utilizes a tri-glide buckle or ladder lock buckle that allows the belt material 104 to slide back and forth to adjust the length and friction holds the belt material 104 once adjusted. In other examples described further herein, the adjustment mechanism 102 can be a mechanical winch system or winch wheel that adjusts the belt material 104. In other examples, the adjustment mechanism 102 can be a lacing system (e.g., cord and pulley, etc.) where the belt material 104 is a cord that can be threaded through eyelets of the adjustment mechanism 102.

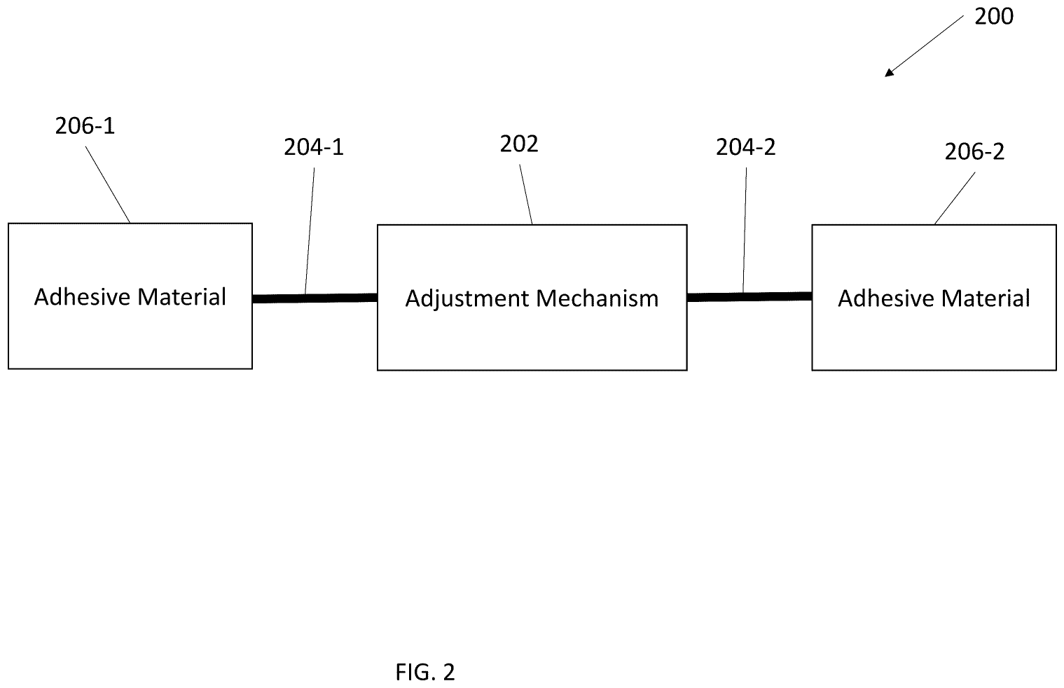

FIG. 2 illustrates an example of a belt adjustment device 200 according to examples described herein. In some embodiments, the belt adjustment device 200 can include similar elements to the belt adjustment device 100 as illustrated in FIG. 1. The belt adjustment device 200 includes a first adhesive material 206-1 coupled to a first portion of belt material 204-1 and a second adhesive material 206-2 coupled to a second portion of belt material 204-2.

The belt adjustment device 200 can include a single adjustment mechanism 202 that can individually adjust and/or simultaneously adjust the first portion of belt material 204-1 and the second portion of belt material 204-2. In this way, the single adjustment mechanism 202 can be utilized to adjust a position of the area of skin coupled to the first adhesive material 206-1 and the area of skin coupled to the second adhesive material 206-2. For example, the first adhesive material 206-1 can be coupled to a first buttock of a human user and the second adhesive material 206-2 can be coupled to a second buttock of the human user. In this example, the single adjustment mechanism 202 can be positioned over a stomach area of the human user to separate the first buttock from the second buttock through the single adjustment mechanism 202 decreasing the length of the first portion of the belt material 204-1 and/or the second portion of the belt material 204-2.

In some embodiments, the adjustment mechanism 202 can include multiple different mechanism described in reference to adjustment mechanism 102 in FIG. 1. For example, the adjustment mechanism 202 can include a first mechanism to adjust the first portion of the belt material 204-1 and a second mechanism to adjust the second portion of the belt material 204-2. In this way, the adjustment mechanism 202 can enable a user to utilize a first system for adjusting the skin portion coupled to the first adhesive material 206-1 and a second system for adjusting the skin portion coupled to the second adhesive material 206-2.

Providing different systems for altering a position of each skin portion coupled to the first adhesive material 206-1 and the second adhesive material 206-2 can enable a user to specifically provide different levels of tension on the corresponding skin portion. In addition, the different systems for altering the position of each skin portion can enable a user to utilize a system with a defined level of accuracy or increments of adjustment for each skin portion. For example, a first skin portion coupled to the first adhesive material 206-1 may require a greater level of accuracy or a more minute level of adjustment compared to a second skin portion coupled to the second adhesive material 206-2. In this way, the first belt 204-1 can be a toothed strap that is fed through a rachet buckle of the adjustment mechanism 202 and the second belt 204-2 can be a standard holed system that is adjusted through a standard buckle of the adjustment mechanism 202.

Thus, the adjustment mechanism 202 can include a first side that can interact with a plurality of different types of belt materials for the first belt 204-1 and a second side that can interact with a plurality of different types of belt materials for the second belt 204-2. In some embodiments, the adjustment mechanism 202 can include a plurality of locations that can be utilized to coupled to different types of belt materials. Although two belts (first belt 204-1, second belt 204-2, etc.) are illustrated herein, adjustment mechanisms 202 with additional coupling locations are also possible. In this way, the angle of the tension between the first adhesive material 206-1 and the second adhesive material 206-2 can be altered. In some embodiments, this can be performed by altering a location of the coupling location between the adjustment mechanism 202 and a corresponding belt (e.g., first belt 204-1, second belt 204-2, multiple other belts, etc.).

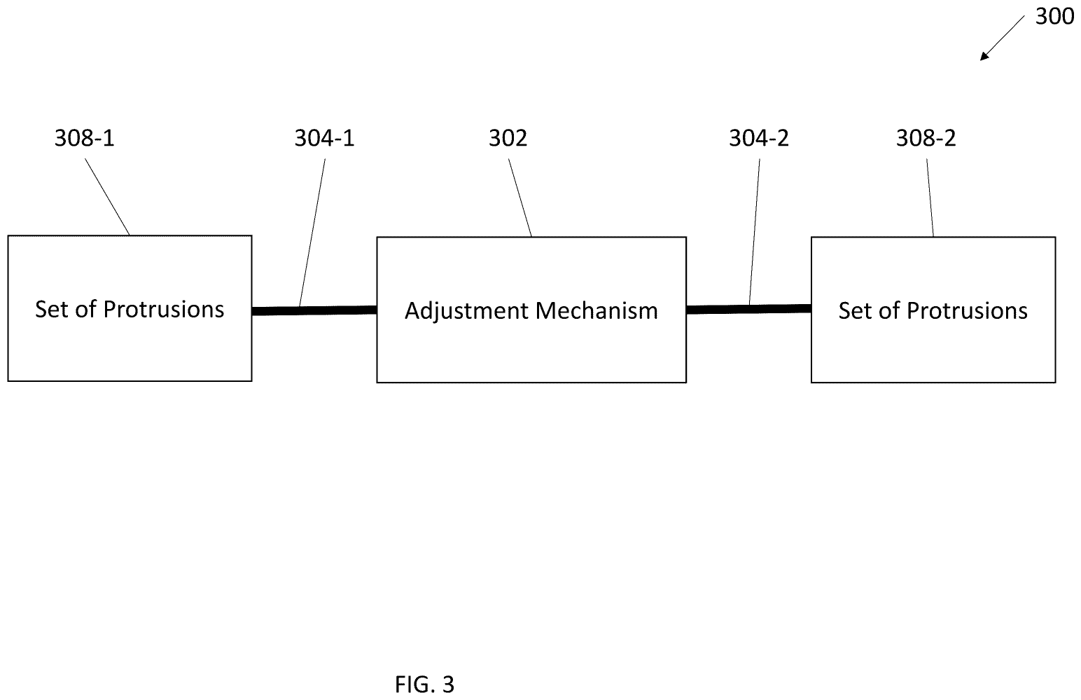

FIG. 3 illustrates an example of a belt adjustment device 300 according to examples described herein. In some embodiments, the belt adjustment device 300 can include similar elements to the belt adjustment device 200 as illustrated in FIG. 2 and/or the elements to the belt adjustment device 100 as illustrated in FIG. 1.

The belt adjustment device 300 includes a first set of protrusions 308-1 coupled to a first portion of belt material 304-1 and a second set of protrusions 308-2 coupled to a second portion of belt material 304-2. In some embodiments, the set of protrusions can be finger or forked structures that can be utilized to grab or couple a portion of skin such as, but not limited to, a buttock of a human user. In some examples, the protrusions can be coated in a material that can allow the protrusions to maintain contact with the human skin. For example, the protrusions can be coated with a polymer material.

The belt adjustment device 300 can include a single adjustment mechanism 302 that can individually adjust and/or simultaneously adjust the first portion of belt material 304-1 and the second portion of belt material 304-2. In this way, the single adjustment mechanism 302 can be utilized to adjust a position of the area of skin coupled to the first set of protrusions 308-1 and the area of skin coupled to the second set of protrusions 308-2. For example, the first set of protrusions 308-1 can be coupled to a first buttock of a human user and the second set of protrusions 308-2 can be coupled to a second buttock of the human user. In this example, the single adjustment mechanism 302 can be positioned over a stomach area of the human user to separate the first buttock from the second buttock through the single adjustment mechanism 302 decreasing the length of the first portion of the belt material 304-1 and/or the second portion of the belt material 304-2.

In some embodiments, the first set of protrusions 308-1 and/or the second set of protrusions 308-2 can be any number of different mechanical devices that can interact with the skin of a human user. The mechanical devices can be devices other than adhesives or devices that can be combined with adhesives. For example, the protrusions can be hook mechanism that are made of a hard material (e.g., stainless steel, medical grade polymers, etc.) that have smooth or rounded edges to grip the skin without puncturing or tearing the skin. The hook mechanism can be a curve, claw-like, or S-shaped to conform with the elasticity and structure of skin. In other embodiments, the protrusions can be mechanical fingers or mechanical clamps that are finger-like projections or jaws that are coated with a soft material or padded material to distribute the pressure of the projections evenly. In some embodiments, the protrusions can be vacuum- assisted hooks or fingers that utilize suction in combination with the mechanical protrusions. Although specific examples, are provided, other types of clastic gripping tools could be utilized.

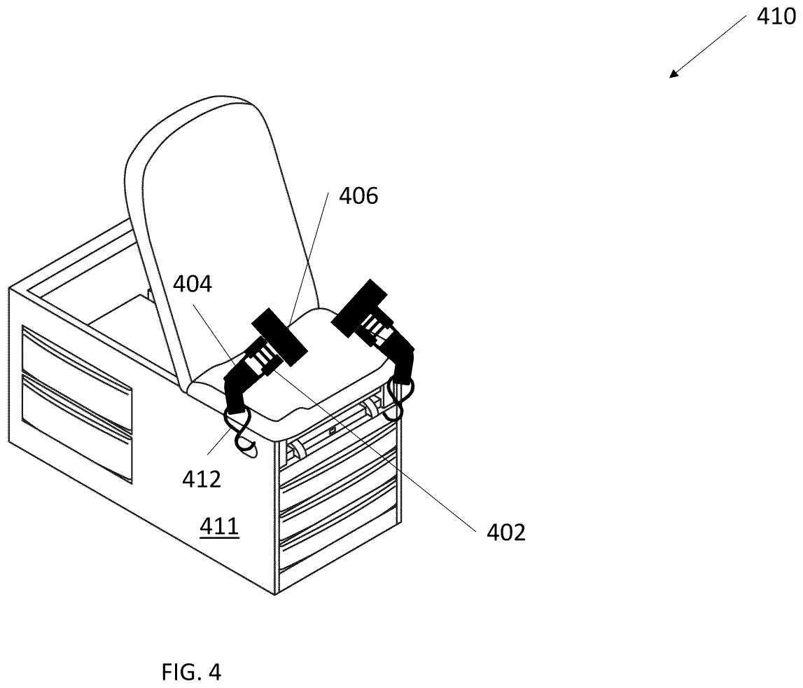

FIG. 4 illustrates an example of system 410 that includes a belt adjustment device utilized with an additional medical device 411 according to examples described herein. The system 410 can include a medical device 411 such as a medical chair or medical table that can allow the belt adjustment devices to be coupled to the medical device 411 on one end and coupled to a human user on the other end. For example, the belt adjustment device can include an attachment device 412 (e.g., hook, clamp, etc.) that can be coupled to the medical device 411.

The belt adjustment devices can include similar elements to the belt adjustment device 200 as illustrated in FIG. 2 and/or the elements to the belt adjustment device 100 as illustrated in FIG. 1. For example, the belt adjustment devices can include a belt portion 404, an adjustment device 402 and/or an adhesive material 406. In these embodiments, the medical device 411 can be utilized as an anchor device to help alter a location of an area of skin coupled to the adhesive material 406 by shortening the length of the belt portion 404 between the attachment device 412 and the adhesive material 406.

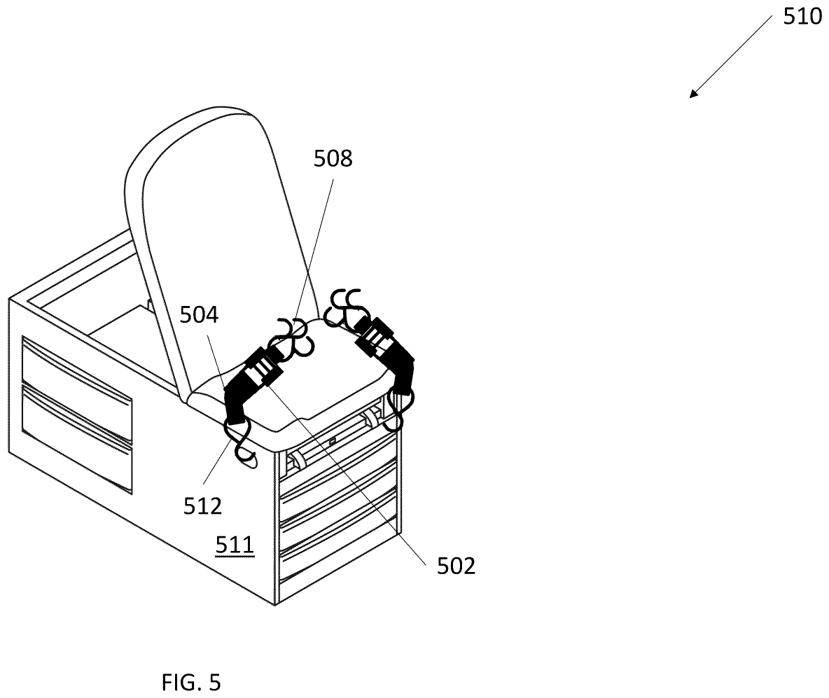

FIG. 5 illustrates an example system 510 that includes a belt adjustment device utilized with an additional medical device 511 according to examples described herein. The system 510 can include the same or similar elements as system 410 as illustrated in FIG. 4. The system 510 can include a medical device 511 such as a medical chair or medical table that can allow the belt adjustment devices to be coupled to the medical device 511 on one end and coupled to a human user on the other end. For example, the belt adjustment device can include an attachment device 512 (e.g., hook, clamp, etc.) that can be coupled to the medical device 511.

The belt adjustment devices can include similar elements to the belt adjustment device 200 as illustrated in FIG. 2 and/or the elements to the belt adjustment device 100 as illustrated in FIG. 1. For example, the belt adjustment devices can include a belt portion 504, an adjustment device 502 and/or a set of protrusions 508. In these embodiments, the medical device 511 can be utilized as an anchor device to help alter a location of an area of skin coupled to the set of protrusions 508 by shortening the length of the belt portion 504 between the attachment device 512 and the set of protrusions 508.

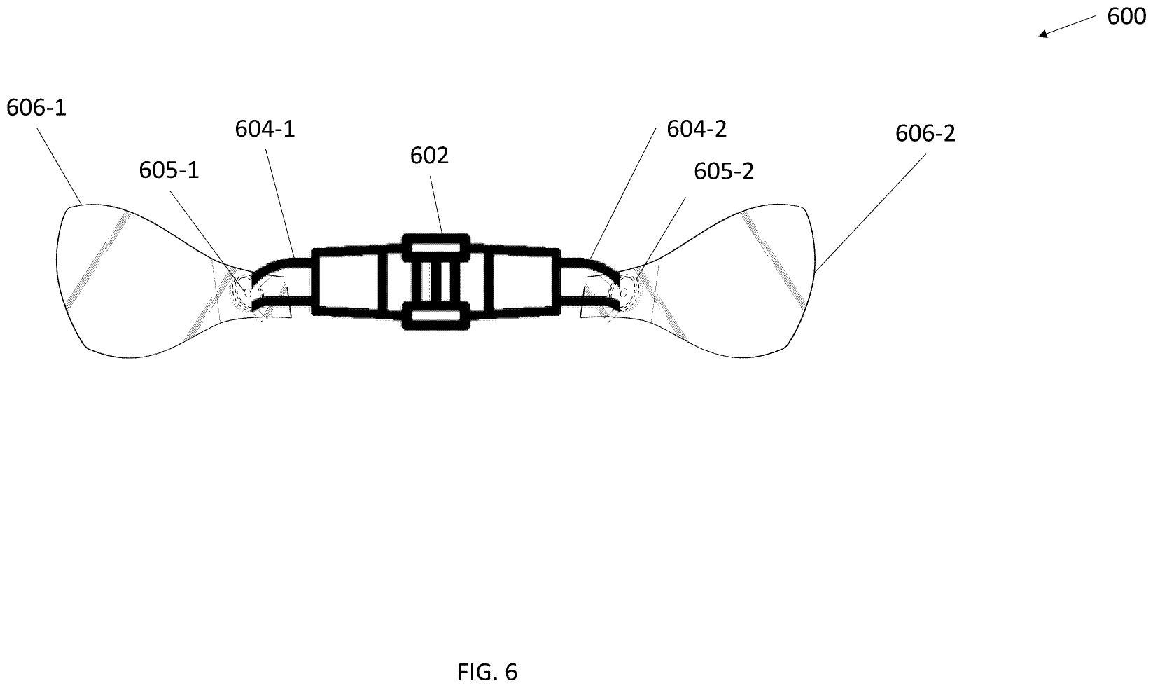

FIG. 6 illustrates an example of a belt adjustment device 600 according to examples described herein. In some embodiments, the belt adjustment device 600 can include the same or similar elements as the belt adjustment device 200 as illustrated in FIG. 2. The belt adjustment device 600 includes a first adhesive material 606-1 coupled to a first portion of belt material 604-1 and a second adhesive material 606-2 coupled to a second portion of belt material 604-2. In some embodiments, the first adhesive material 606-1 and the second adhesive material 606-2 can be adhesive pads that include an adhesive on a first surface and a connector interface 605-1, 605-2 on a second surface (e.g., opposite surface of the first surface, etc.).

The belt adjustment device 600 can include a single adjustment mechanism 602 that can individually adjust and/or simultaneously adjust the first portion of belt material 604-1 and the second portion of belt material 604-2. In this way, the single adjustment mechanism 602 can be utilized to adjust a position of the area of skin coupled to the first adhesive material 606-1 and the area of skin coupled to the second adhesive material 606-2. For example, the first adhesive material 606-1 can be coupled to a first buttock of a human user and the second adhesive material 606-2 can be coupled to a second buttock of the human user. In this example, the single adjustment mechanism 602 can be positioned over a stomach area of the human user to separate the first buttock from the second buttock through the single adjustment mechanism 602 decreasing the length of the first portion of the belt material 604-1 and/or the second portion of the belt material 604-2.

In some embodiments, the first portion of the belt material 604-1 can be connected to the first adhesive material 606-1 through a connector interface 605-1. In addition, the second portion of the belt material 604-2 can be connected to the second adhesive material 606-2 through a connector interface 605-2. In these embodiments, the first portion of the belt material 604-1 can be connected to and disconnected from the first adhesive material 606-1. In some embodiments, the connector interface 605-1, 605-2 can be a quick connect/quick disconnect connector that can couple the first portion of the belt material 604-1. In this way, the first adhesive material 606-1 can remain connected to the human skin of the user so the user doesn't have to remove the first adhesive material 606-1 after each use of the adjustment device. For example, the first adhesive material 606-1 and the second adhesive material 606-2 can be attached to the skin portions of the human user separate from the adjustment mechanism 602 and/or the first portion of the belt material 604-1 or the second portion of the belt material 604-2.

In some embodiments, the connector interface 605-1 between the first portion of the belt material 604-1 and the first adhesive material 606-1 or the connector interface 605-2 between the second portion of the belt material 604-2 and the second adhesive material 606-2 can be an emergency release function. That is, a user can release the first portion of the belt material 604-1 from the first adhesive material 606-1 in case of an emergency.

In some embodiments, the connector interface 605-1, 605-2 can be a clip and slot mechanism that utilizes spring loaded lip at the belt material and a slot or loop on the surface of the adhesive material. In other embodiments, the connector interface 605-1, 605-2 can be a snap button connection that includes a snap button receive (e.g., female socket) coupled to the adhesive material or adhesive pad and the belt material can include a snap button stud (e.g., male socket, etc.). In some embodiments, the connector interface 605-1, 605-2 can include a button or mechanism that can be depressed to decouple the adhesive material from the belt material. In these embodiments, the button can be released to couple the adhesive material to the belt material.

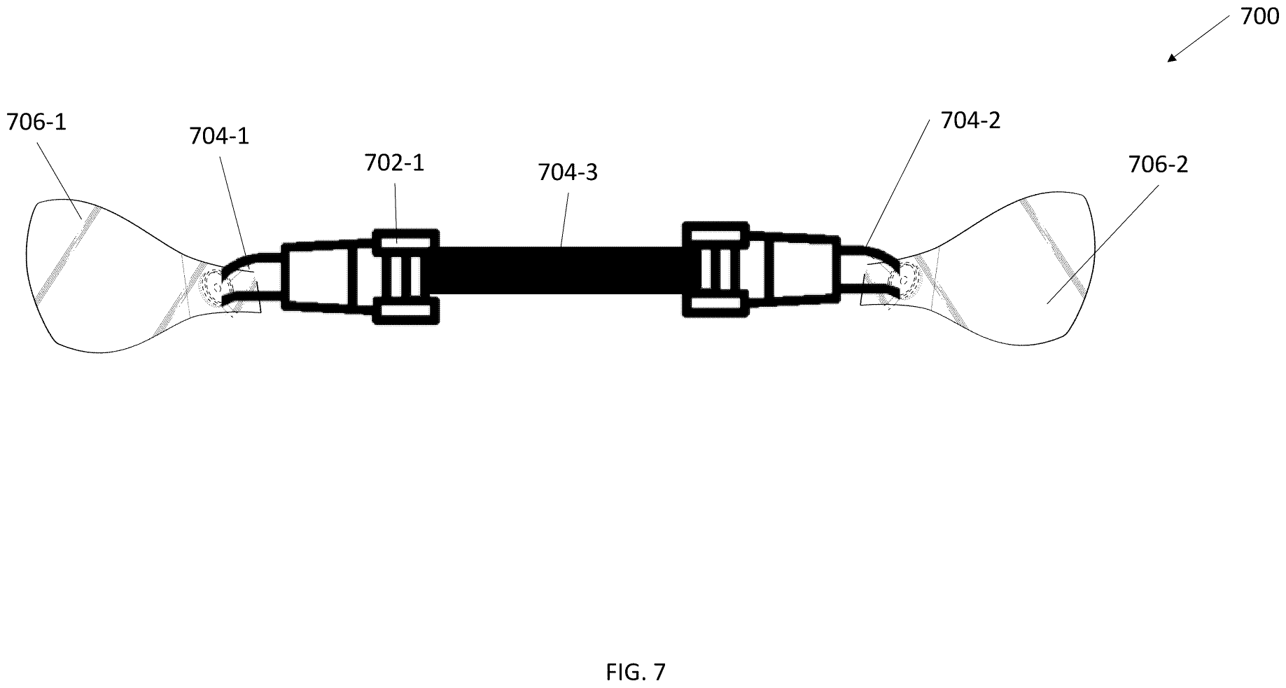

FIG. 7 illustrates an example of a belt adjustment device 700 according to examples described herein. In some embodiments, the belt adjustment device 700 can include the same or similar elements as the belt adjustment device 200 as illustrated in FIG. 2 and/or the belt adjustment device 600 as illustrated in FIG. 6. The belt adjustment device 700 includes a first adhesive material 706-1 coupled to a first portion of belt material 704-1 and a second adhesive material 706-2 coupled to a second portion of belt material 704-2.

The belt adjustment device 700 can include a first adjustment mechanism 702-1 that can be coupled to a second adjustment mechanism 702-2 by a third belt material 704-3. In these embodiments, the first adjustment mechanism 702-1 can adjust the first portion of belt material belt material 704-1 and the second adjustment mechanism 702-2 can be utilized to adjust the second portion of belt material 704-2. In this way, the belt adjustment device 700 can be utilized to individually adjust a position of the area of skin coupled to the first adhesive material 706-1 and the area of skin coupled to the second adhesive material 706-2. For example, the first adhesive material 706-1 can be coupled to a first buttock of a human user and the second adhesive material 706-2 can be coupled to a second buttock of the human user. In this example, the third belt material 704-3 can be positioned over a stomach area of the human user to allow access to the first adjustment mechanism 702-1 and/or second adjustment mechanism 702-2. In this way, the first adjustment mechanism 702-1 and second adjustment mechanism 702-2 can be utilized to individually separate the first buttock from the second buttock by individually decreasing the length of the first portion of the belt material 704-1 and/or the second portion of the belt material 704-2. This can allow for more precise pressure to be applied to the first adhesive material 706-1 and/or second adhesive material 706-2.

In the foregoing detailed description of the disclosure, reference is made to the accompanying drawings that form a part hereof, and in which is shown by way of illustration how examples of the disclosure may be practiced. These examples are described in sufficient detail to enable those of ordinary skill in the art to practice the examples of this disclosure, and it is to be understood that other examples may be utilized and that process, electrical, and/or structural changes may be made without departing from the scope of the disclosure.

The figures herein follow a numbering convention in which the first digit corresponds to the drawing figure number and the remaining digits identify an element or component in the drawing. For example, reference numeral 102 may refer to element 102 in FIG. 1 and an analogous element may be identified by reference numeral 302 in FIG. 3. Elements shown in the various figures herein can be added, exchanged, and/or eliminated to provide additional examples of the disclosure. In addition, the proportion and the relative scale of the elements provided in the figures are intended to illustrate the examples of the disclosure and should not be taken in a limiting sense.

It can be understood that when an element is referred to as being “on,” “connected to”, “coupled to”, or “coupled with” another element, it can be directly on, connected, or coupled with the other element or intervening elements may be present. In contrast, when an object is “directly coupled to” or “directly coupled with” another element it is understood that are no intervening elements (adhesives, screws, other elements) etc. Further, as used herein, “a” refers to one such thing or more than one such thing.

What is claimed is:

1. A device, comprising:
a belt;
an adjustment mechanism to adjust a distance of the belt between an end of the belt and the adjustment mechanism;
an adhesive material coupled to the end of the belt to adhere to a portion of skin of a human user, wherein the adjustment mechanism adjusts a distance between the portion of skin of the human user and the adjustment mechanism.

2. The device of claim 1, wherein the adhesive material is removable from the end of the belt.

3. The device of claim 1, wherein the adhesive material is coupled to the end of the belt by a connection interface.

4. The device of claim 3, wherein the connection interface is a system of hooks and loops.

5. The device of claim 1, wherein the adjustment mechanism is a ratcheting mechanism to adjust the distance of the belt incrementally.

6. A device, comprising:
a belt;
an adjustment mechanism to adjust a distance of the belt between a first end and a second end;
a first adhesive material coupled to the first end of the belt to adhere to a first portion of skin of a human user; and
a second adhesive material coupled to the second end of the belt to adhere to a second portion of skin of human user, wherein the adjustment mechanism adjusts a distance between the first portion of skin of the human user and the second portion of skin of the human user.

7. The device of claim 6, wherein the first adhesive material coupled to the belt is applied directly to the first end and the second adhesive material coupled to the belt is applied directly to the second end of the belt.

8. The device of claim 6, wherein the first adhesive material is applied to a first disposable surface and the second adhesive material is applied to a second disposable surface.

9. The device of claim 6, wherein the first end and second end include a hooking apparatus to interact with the skin of the human user.

10. A device, comprising:
a belt;
an adjustment mechanism to adjust a distance of the belt between a first end and a second end;
a first set of protrusions coupled to the first end of the belt to interact with a first portion of skin of a human user; and
a second set of protrusions coupled to the second end of the belt to interact with a second portion of skin of the human user, wherein the adjustment mechanism adjusts a distance between the first portion of skin of the human user and the second portion of skin of the human user.

11. The device of claim 10, further comprising a first plurality of caps to cover the first set of protrusions and a second plurality of caps to cover the second set of protrusions.

12. The device of claim 11, wherein the caps are attachable and removable from the protrusions.

13. The device of claim 11, wherein the caps consist of an outer adhesive surface.

14. The device of claim 10, wherein the protrusions are no more than 4.5 inches in length.

15. The device of claim 10, wherein the adjustment mechanism includes an attachment mechanism to couple the adjustment mechanism to a base.
